(19) United States
(12) Patent Application Publication
(10) Pub. No.: US 2006/0223175 A1
(43) Pub. Date: Oct. 5, 2006

(54) Bioreactor for cultivating tissue cells

(75) Inventors: Yu-Chen Hu, Hsinchu (TW); Huang-Chi Chen, Hsinchu (TW); Chun-Jen Liao, Taipei (TW)

Correspondence Address: PAI PATENT & TRADEMARK LAW FIRM, 1001 FOURTH AVENUE, SUITE 3200, SEATTLE, WA, 98154, US

(73) Assignee: National Tsing Hua University, Hsinchu (TW)

(21) Appl. No.: 11/176,411

(22) Filed: Jul. 7, 2005

(30) Foreign Application Priority Data
Apr. 4, 2005 (TW) 94110760

(51) Int. Cl. C12M 3/00

(52) U.S. Cl. 435/298.1; 435/299.1

(57) ABSTRACT

A bioreactor for cultivating tissue cells comprises a vessel containing a gas phase and a liquid phase therein, at least a substrate on which the tissue cells are attached, and a movable shaft to which the substrate is fixed. The movable shaft carries the substrate into and out of the gas and liquid phases so as to apply shear stress to the tissue cells.

### BACKGROUND OF THE INVENTION

a) Field of the Invention

The invention relates to a bioreactor and, more particularly, to a bioreactor able to apply shear stress to cultivated tissue cells.

b) Description of the Related Art

In recent years, as biotechnology develops, technologies for cultivating tissue cells in vitro are getting more and more attention. In order to supply sufficient nutrition and air for tissue cells, various bioreactors and cultivating devices have been designed. With these bioreactors, a great amount of tissue cells can be efficiently cultivated in a short time period so as to meet the requirements for the related research and development.

When a bioreactor for cell culture is designed, nutrient transfer and air exchange are the two chief considerations. On the other hand, in addition to sufficient nutrition and air, certain specific mechanical stimulations are essential for some differentiated tissues in the human body, such as cartilage, to grow rapidly and maintain their phenotypes.

Among the numerous bioreactor designs, a spinner flask is one kind of the most popular bioreactors and its working principle is that a turbulent region is formed by stirring the fluid in the reactor, which is caused by magnetic force, so that the air and nutrient in the reactor can be mixed. However, since the air exchange is merely conducted at the interface between the air and the liquid, the effect of air mass transfer is restricted. Furthermore, if tissues are cultivated in a spinner flask, dense cell layers will be formed on the exterior of the cultivated tissues and the inner cells will die from deficiency of air and nutrient. Besides, although a spinner flask can provide mechanical stimulations such as shear stress, it is hard to control the magnitude of the shear stress applied to the tissue cells, and thus that is unfavorable to cell growth.

Another prevalent bioreactor is the rotating-wall vessel bioreactor, which can provide a random and low shear stress for the tissue cells attached on a rotatable wall of the bioreactor through rotation thereof. It has frequently been implemented to culture tissue-engineered cartilage, but the cartilage usually grows loosely and unevenly. Besides, scientists have developed a method for cultivating tissue cells in a column into which a liquid growth medium is fed along the axial direction. According to this method, medium is perfused through the cell/substrate constructs to provide medium exchange and induce columnar cell orientation and matrix assembly, yet the propagating cells occupy the space in the column and nutrient limitation in the inner region occurs during the late growth stage.

To meet the requirement of mechanical stimulation, other reactor systems that provide oscillatory mechanical compression, fluid-induced shear, cyclic hydrostatic fluid pressure, and hydrodynamic loading have been developed. However, none of these bioreactors can provide an environment with sufficient nutrient transfer and air exchange. Additionally, in order to increase dissolved oxygen (DO), the present bioreactors should further comprise an oxygen exchange system and hence the cost for cell culture increases.

In view of the above, a bioreactor that is capable of providing cultivated cells with nutrient, air, and mechanical stimulation sufficiently and uniformly will greatly enhance the efficiency of cultivating tissue cells in vitro.

### SUMMARY

Therefore, an object of the present invention is to provide a bioreactor system capable of supplying sufficient nutrient, air, and mechanical stimulation for cultivated cells so as to cultivate tissue cells in vitro efficiently.

A bioreactor system for cultivating tissue cells according to the invention comprises a vessel containing a gas phase and a liquid phase, at least one substrate on which the tissue cells are attached, and a movable shaft. The substrate is fixed onto the shaft, and the movable shaft carrying the substrate into and out of the gas and liquid phases so as to apply shear stress to the tissue cells.

In one aspect of the invention, the movable shaft is a rotatable shaft disposed parallel to the surface of the liquid phase, which carries the substrate into and out of the gas and liquid phases by its rotation.

In another aspect of the invention, the movable shaft is an oscillating shaft disposed above the surface of the liquid phase, which carries the substrate into and out of the gas and liquid phases by its oscillation.

In still another aspect of the invention, the movable shaft is a reciprocating shaft able to move perpendicularly to the surface of the liquid phase, which carries the substrate into and out of the gas and liquid phases by its reciprocation.

By controlling movement of the shaft, the bioreactor system of the invention not only provide mild mechanical stimulation while avoiding excessive damage to cells, but also achieve sufficient nutrient transfer and air exchange.

### BRIEF DESCRIPTION OF THE DRAWINGS

FIG. 1 is a schematic diagram illustrating a bioreactor according to the first embodiment of the invention.

FIG. 2 is a schematic diagram illustrating a bioreactor according to the second embodiment of the invention.

FIG. 3 is a schematic diagram illustrating a bioreactor according to the third embodiment of the invention.

FIG. 4 schematically shows that substrates are fixed on a shaft through stainless steel baskets or plastic baskets.

FIG. 5 shows the periodic relationship between mean shear stress acting on a substrate and the position of the substrate when the shaft of the bioreactor shown in FIG. 1 rotates counterclockwise at a speed of 10 rpm (0-π represents gas phase while π-2π represents liquid phase).

FIG. 6 shows the sectioned samples from (A) 4-week 2R10/2 culture in the bioreactor shown in FIG. 1, (B) articular cartilage of a 7-day-old rat, and (C) 4-week spinner culture.

### DETAILED DESCRIPTION OF THE INVENTION

According to the invention, a bioreactor mainly comprises a vessel containing a gas phase (air) and a liquid phase (medium), at least a substrate on which the tissue cells are attached, and a movable shaft to which the substrate is fixed. The movable shaft carries the substrate into and out of the gas and liquid phases so as to apply shear stress to the tissue cells. Several embodiments will be described as follows to clearly explain the above structure.

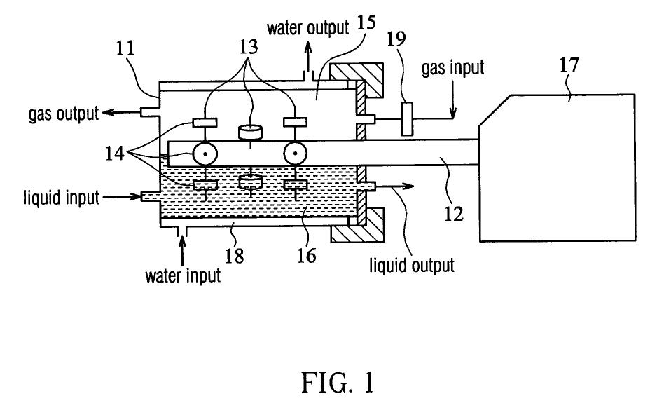

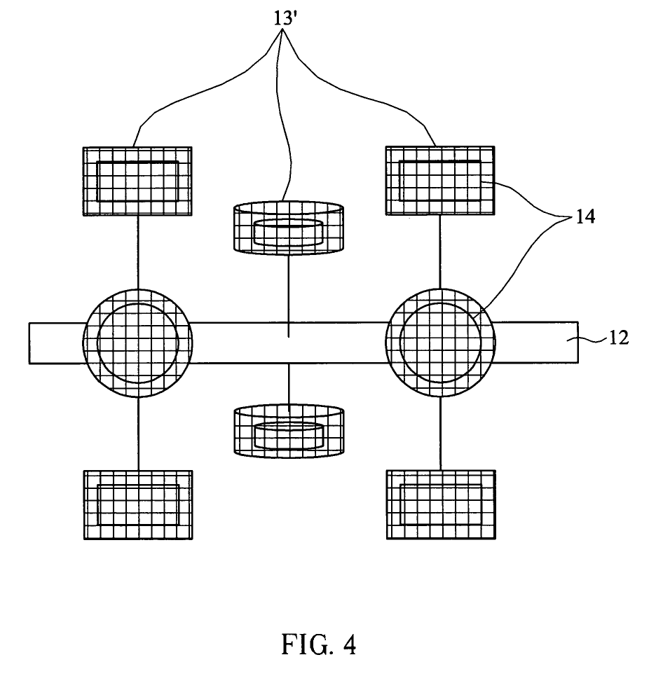

FIG. 1 is a schematic diagram illustrating a bioreactor according to the first embodiment of the invention. In this embodiment, the main body of the bioreactor is a cylindrical vessel 11 containing gas 15 and liquid 16 that are essential for cell growth. For example, gas 15 can be air or a gaseous mixture including 2%-20% carbon dioxide, and liquid 16 can be a common liquid growth medium. For convenient replacement and sampling, the cylindrical vessel 11 may include some ports, through which gas 15 and liquid 16 can be supplied from or discharged to external devices, such as a medium reservoir or an incubator, and an air filter 19 can be installed in front of the inlet port of gas to filter out contaminants in the supplied gas. Besides, the bioreactor further comprises a rotatable shaft 12, which is parallel to the surface of liquid 16. The rotation speed of the shaft 12 is precisely controlled by a driving device 17, such as a peristaltic pump, a reciprocating pump, or a motor etc. At least one substrate 14 on which cultivated tissue cells are attached is fixed to the rotatable shaft 12. In this embodiment, the substrate 14 is positioned using a stainless steel needle 13 soldered on the shaft 12. However, it can be fixed to the shaft 12 by other means, for example, as shown in FIG. 4, by a stainless steel or a plastic basket 13′. The substrate 14 can be composed of a porous or a biocompatible material. Furthermore, a temperature-controlling system 18, such as a water jacket, can be disposed around the cylindrical vessel 11 to control the temperature of the bioreactor. In order to increase the gas/liquid mass transfer rate, the rotatable shaft 12 can be further provided with impellers.

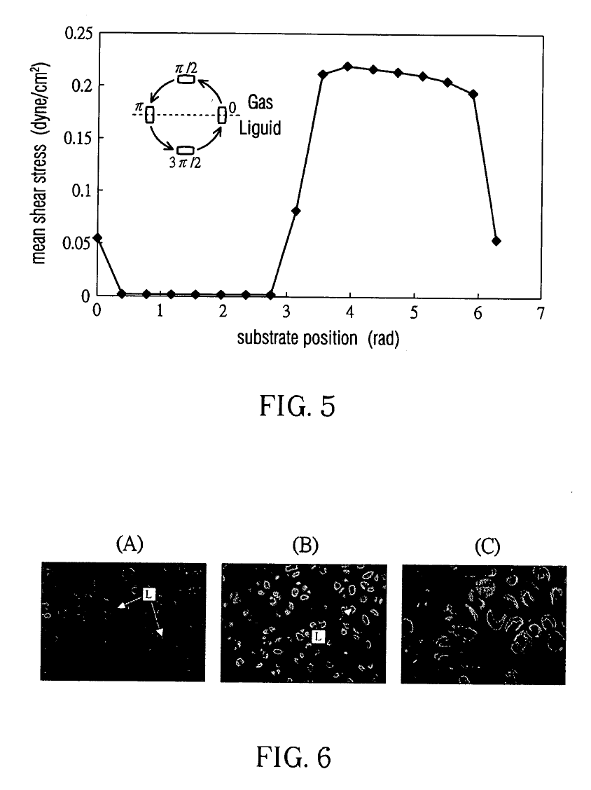

By means of rotation of the shaft 12, the substrate 14 fixed on it periodically moves into and out of gas 15 and liquid 16 so as to apply shear stress to tissue cells attached on the substrate 14. For estimating and precisely controlling shear stress acting on the substrate 14 to promote uniform cell growth while avoiding excessive damage to cells, the spatial distribution of shear stress exerting on the substrate 14 is simulated using FLUENT (Fluent Corp.). The method for analyzing shear stress has been disclosed in the applicants' article, “A Novel Rotating-Shaft Bioreactor for Two-Phase Cultivation of Tissue-Engineered Cartilage”, Biotechnol. Prog., 2004, Vol. 20, 1802-1809, which is incorporated by reference. FIG. 5 shows the periodic relationship between mean shear stress acting on a substrate and the position of the substrate when the rotatable shaft 12 of the bioreactor rotates counterclockwise at a speed of 10 rpm, wherein the position of the substrate is represented as its angle (in radian) relative to the rotatable shaft 12 (0-π represents gas phase while π-2π represents liquid phase). As shown in FIG. 5, mean shear stress acting on tissue cells periodically varies with rotation of the substrate from 0 to 0.21 dyn/cm², thus ensuring that the bioreactor creates a mild yet dynamic microenvironment. Moreover, according to the simulation result, the maximal shear stress exerting on tissue cells is approximately linearly proportional to the rotating speed of the shaft 12. As a result, shear stress exerting on tissue cells can be precisely adjusted by controlling rotation of the shaft 12.

On the other hand, during rotation of the shaft 12, since tissue cells attached on the substrate 14 alternately contact with gas 15 and liquid 16 and perform gas and nutrient exchange, they can obtain sufficient oxygen and nutrient without an additional oxygen exchange system.

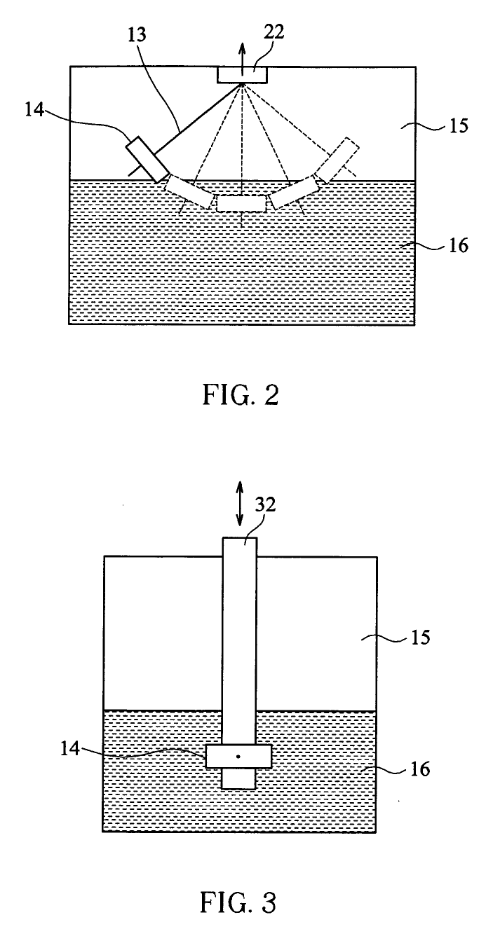

FIG. 2 is a schematic diagram illustrating a bioreactor according to the second embodiment of the invention. As shown in FIG. 2, the bioreactor in this embodiment is substantially the same as that in the first embodiment except that the rotatable shaft 12 in the first embodiment is replaced with an oscillating shaft 22 disposed above the surface of liquid 16. Similarly, a substrate 14 is fixed to the oscillating shaft 22 through a stainless steel needle 13. By means of oscillation of the shaft 22, the substrate 14 periodically moves into and out of gas 15 and liquid 16 so as to apply shear stress to tissue cells attached on the substrate 14. In addition, shear stress exerting on tissue cells can be properly adjusted by controlling oscillation of the shaft 22.

FIG. 3 is a schematic diagram illustrating a bioreactor according to the third embodiment of the invention. As shown in FIG. 3, the bioreactor in this embodiment is substantially the same as that in the first embodiment except that the rotatable shaft 12 in the first embodiment is replaced with a reciprocating shaft 32 that is able to move perpendicularly to the surface of liquid 16. Likewise, a substrate 14 is fixed to the reciprocating shaft 32 through a stainless steel needle (represented as a point in FIG. 3). By means of reciprocation of the shaft 32, the substrate 14 periodically moves into and out of gas 15 and liquid 16 so as to apply shear stress to tissue cells attached on the substrate 14. Also, shear stress exerting on tissue cells can be properly adjusted by controlling reciprocation of the shaft 32.

### EXAMPLE

In the following example, the bioreactor in the first embodiment was used to cultivate chondrocytes for demonstrating the effects of the bioreactors disclosed by the invention. Besides, it should be noted that the materials, operation conditions, and analytical methods etc. have been specifically described in the Applicants' article, “A Novel Rotating-Shaft Bioreactor for Two-Phase Cultivation of Tissue-Engineered Cartilage”, Biotechnol. Prog., 2004, Vol. 20, 1802-1809, which is incorporated by reference in their entirety.

At first, chondrocytes, isolated from the articular cartilages of 7-day-old Wister rats, were seeded onto porous poly(L-lactide-co-glycolide) (PLGA) scaffolds (the substrates) in spinner flasks for three days (seeding density: 3×10⁶ cells/scaffold). Then, the chondrocyte/scaffold constructs were transferred into the bioreactor shown in FIG. 1 and fixed to the rotatable shaft 12 by being threaded and positioned on the stainless steel needles 13. Approximately half of the cylindrical vessel 11 space was filled with a liquid growth medium, and humidified gas (37° C., 5% CO₂) passing through the air filter 19 (0.22 μm) previously was introduced into the cylindrical vessel 11. Furthermore, the temperature in the bioreactor system was controlled at 37° C. by the water circulating through the water jacket.

Thereafter, the chondrocyte/scaffold constructs were cultivated in the bioreactor for 4 weeks with medium and gas perfusion while under different rotating speeds (2, 5, and 10 rpm) of the shaft 12, and the cultures are denoted as R2, R5, and R10 cultures, respectively. For comparison, the constructs were also cultivated in spinner flasks operating at 50 rpm, a speed commonly used for cartilage cultivation. Finally, constructs were taken out and analyzed to determine the results of cell proliferation, extra-cellular matrix (ECM) biosynthesis, and cell metabolism, which are shown in Table 1.

TABLE 1
Chondrocyte proliferation, metabolism, matrix biosynthesis, and
GAG release of the constructs cultivated in different culture
conditions for 4 weeks^{a}.

| | Spinner | R2 | R5 | R10 |
|---|---|---|---|---|
| Cell number/scaffold | 7.4 ± 0.5 | 7.8 ± 0.3 | 8.0 ± 0.1 | 7.0 ± 0.2 |
| (10⁶) | | | | |
| Y_{L/G}^{b} | 1.52 | 1.80 | 1.79 | 1.26 |
| COL (dw %)^{c} | 7.1 ± 0.3 | 6.1 ± 0.8 | 5.0 ± 0.5 | 10.8 ± 1.7 |
| COL (mg)/construct | 4.1 ± 0.5 | 2.9 ± 0.3 | 2.8 ± 0.2 | 5.9 ± 0.5 |
| GAG (dw %) | 3.1 ± 0.3 | 2.6 ± 0.2 | 2.9 ± 0.3 | 1.4 ± 0.1 |
| GAG (mg)/construct | 1.8 ± 0.2 | 1.2 ± 0.2 | 1.6 ± 0.2 | 0.8 ± 0.1 |
| GAG release (mg)/ | 6.5 ± 1.5 | 4.8 ± 1.7 | 5.1 ± 1.2 | 28.2 ± 5.0 |
| construct^{d} | | | | |

^{a}The data of cell number, collagen (COL) and glycosaminoglycan (GAG) represent mean ± SD of two independent experiments.
^{b}The average molar ratio of lactate production to glucose consumption over 4 weeks.
^{c}The dry weight percentage of collagen.
^{d}Cumulative amount of GAG released into the medium over 4 weeks.

As shown in Table 1, the chondrocyte numbers per scaffold after 4 weeks exhibited small variations [(7-8)×10⁶ cells] for all cultures, indicating that cell proliferation was independent of rotating speed and culture vessel. In contrast, the average values of the molar ratio of lactate production to glucose consumption (Y_{L/G}≈1.8) over 4 weeks in R2 and R5 cultures were higher than that in spinner culture (≈1.52) but were efficiently lowered to ≈1.26 by increasing the rotating speed to 10 rpm (R10). Y_{L/G} has been used as an indicator of the cell metabolism, whereby a value approaching 2 indicates an anaerobic metabolism. The high Y_{L/G} (≈1.8) in R2 and R5 cultures thus suggested a relatively anaerobic metabolism at low speeds. Nonetheless, increasing the rotating speed to 10 rpm (R10) successfully enhanced oxygen transfer and thus switched the metabolism to be more aerobic.

On the other hand, collagen (COL) and glycosaminoglycan (GAG) are the main ECMs of articular cartilage and ECM synthesis also reflected the switch in the metabolic pathway. As shown in Table 1, collagen synthesis in R10 culture (5.9 mg per construct) was about 100% and 117% higher than in R2 and R5 cultures, suggesting that higher rotating speed more effectively stimulated the collagen synthesis. Besides, although GAG content (0.8 mg/construct) in R10 culture was about 50% and 100% lower than in R2 and R5 cultures, meanwhile, GAG release (28.2 mg) in R10 culture was significantly higher than in other cultures. That proves that higher rotating speed resulted in more GAG synthesis, but less GAG accumulation, probably because of the GAG release into the medium.

Although higher rotating speed suppressed GAG deposition in the constructs, this situation can be improved by a two-stage culture strategy. For example, to enhance the GAG retention in the construct, the rotating speed of the shaft 12 was maintained at 10 rpm for the first 3 weeks but was lowered to 2 rpm in week 4, while all other conditions remained identical to those in R10. The culture is denoted as R10/2. To further enhance the ECM synthesis, another culture was operated in a way similar to R10/2, except that the seeding density was doubled to 6×10⁶ cells/scaffold. The culture is denoted as 2R10/2. Also, the results were shown in Table 2, in which the spinner flasks and R10 cultures as described in Table 1 were repeated as controls.

TABLE 2
Properties of 4-week constructs cultured in
the RSB under different conditions^{a}.

| | Spinner | R10 | R10/2 | 2R10/2 |
|---|---|---|---|---|
| Wet weight | 222 ± 30 | 191 ± 22 | 207 ± 25 | 240 ± 32 |
| (mg) | | | | |
| Dry weight | 58.0 ± 1.7 | 54.6 ± 1.8 | 55.0 ± 2.0 | 61.7 ± 1.5 |
| (mg) | | | | |
| GAG (mg)/ | 1.8 ± 0.2 | 0.8 ± 0.1 | 1.6 ± 0.3 | 3.1 ± 0.8 |
| construct | | | | |
| COL (mg)/ | 4.1 ± 0.5 | 5.9 ± 0.5 | 4.7 ± 0.6 | 7.0 ± 0.4 |
| construct | | | | |
| GAG (dw %) | 3.1 ± 0.3 | 1.4 ± 0.1 | 2.9 ± 0.3 | 5.0 ± 0.8 |
| COL (dw %) | 7.1 ± 0.3 | 10.8 ± 1.7 | 8.5 ± 1.0 | 11.3 ± 1.0 |

^{a}The data represent mean ± SD of two independent experiments.

As shown in Table 2, in comparison with R10, R10/2 resulted in about 100% increase in GAG content at week 4, demonstrating the success by lowering rotating speed at later stage of the culture. Doubling the seeding cell density (2R10/2) further improved the collagen synthesis and GAG deposition in comparison with R10/2. That proves that increasing seeding cell density and strategic change in rotating speed at week 3 effectively stimulated cartilage growth and ECM deposition.

Moreover, the cartilage-like constructs were further sectioned and subjected to histological examination. FIG. 6 shows the sectioned samples from (A) 4-week 2R10/2 culture in the bioreactor shown in FIG. 1, (B) articular cartilage of a 7-day-old rat, and (C) 4-week spinner culture. FIG. 6 reveals a striking similarity between the 4-week constructs from 2R10/2 culture (A) and the native rat articular cartilage (B) in terms of cell volume, spatial distribution, and morphology. In contrast, the 4-week constructs from spinner culture (C) exhibited enlarged cell volume and distinct cell morphology, which was indicative of hypertrophy.

According to the results of the above example, shear stress acting on tissue cells cultivated in the bioreactor of the invention can be properly adjusted by controlling movement (e.g. rotation, oscillation, or reciprocation) of the movable shaft, so as to provide mild mechanical stimulation while avoiding excessive damage to cells. Besides, since tissue cells alternately contact with gas and liquid and perform gas and nutrient exchange during movement of the shaft, they can obtain sufficient oxygen and nutrient. Therefore, the bioreactor of the invention can greatly enhance the efficiency of cultivating tissues such as cartilage in vitro.

While the invention has been described by way of example and in terms of the preferred embodiment, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A bioreactor system for cultivating tissue cells comprising:
a vessel containing a gas phase and a liquid phase;
at least one substrate on which the tissue cells are attached; and
a movable shaft to which the substrate is fixed, the movable shaft carrying the substrate into and out of the gas and liquid phases so as to apply shear stress to the tissue cells.

2. The bioreactor system as described in claim 1, wherein the gas phase contains gas essential for cell growth.

3. The bioreactor system as described in claim 1, wherein the liquid phase contains liquid growth medium.

4. The bioreactor system as described in claim 1, wherein the vessel includes a plurality of ports, through which the gas and liquid phases are supplied from or discharged to external devices, respectively.

5. The bioreactor system as described in claim 1, wherein the substrate consists of a porous material.

6. The bioreactor system as described in claim 1, wherein the substrate consists of a biocompatible material.

7. The bioreactor system as described in claim 1, wherein the movable shaft is a rotatable shaft disposed parallel to the surface of the liquid phase, which carries the substrate into and out of the gas and liquid phases by its rotation.

8. The bioreactor system as described in claim 1, wherein the movable shaft is an oscillating shaft disposed above the surface of the liquid phase, which carries the substrate into and out of the gas and liquid phases by its oscillation.

9. The bioreactor system as described in claim 1, wherein the movable shaft is a reciprocating shaft able to move perpendicularly to the surface of the liquid phase, which carries the substrate into and out of the gas and liquid phases by its reciprocation.

10. The bioreactor system as described in claim 1, wherein the substrate is fixed to the movable shaft through a stainless steel needle, a stainless steel basket, or a plastic basket.

11. The bioreactor system as described in claim 1, further comprising a driving device for controlling movement of the movable shaft to adjust shear stress acting on the tissue cells.

12. The bioreactor system as described in claim 11, wherein the driving device is a peristaltic pump, a reciprocating pump, or a motor.

13. The bioreactor system as described in claim 1, wherein the tissue cells are animal cells.

14. The bioreactor system as described in claim 1, wherein the movable shaft is provided with at least one impeller.

15. The bioreactor system as described in claim 1, further comprising a temperature-controlling system for controlling the temperature of the bioreactor system.
